(19) United States
(12) Patent Application Publication
(10) Pub. No.: US 2004/0122482 A1
(43) Pub. Date: Jun. 24, 2004

(54) Nerve proximity method and device

(76) Inventors: James Tung, Nepean (CA); Nima Nourhaghighi, Toronto (CA); Krishan Shah, Mississauga (CA)

Correspondence Address: OGILVY RENAULT, 1981 MCGILL COLLEGE AVENUE, SUITE 1600, MONTREAL, QC, H3A2Y3, CA

(21) Appl. No.: 10/323,672

(22) Filed: Dec. 20, 2002

(51) Int. Cl. A61N 1/18

(52) U.S. Cl. 607/48

(57) ABSTRACT

A method and apparatus are disclosed for proximity detection and confirmation of treatment of a target nerve such as the facet nerve. The apparatus includes a probe that can deliver electrical current for stimulation of the facet nerve, and physiological sensors for transduction of multifidus muscle activity. A generator produces electrical current at physiological stimulation frequencies, and physiological sensor signals of muscle activity are displayed, analyzed and recorded with the use of a measuring device. A method using stimulation-induced multifidus activity to localize and confirm treatment of the facet nerve is disclosed.

### TECHNICAL FIELD

The invention relates to a method and apparatus for the localization, treatment and confirmation of treatment of a nerve, particularly of the medial branch of the dorsal ramus nerve (facet nerve).

### BACKGROUND OF THE ART

In 1988, Boachie-Adjei reported that low back pain affected approximately 70% of the population in industrialized countries. Wall and Melzack have described low back pain as the most frequent cause of activity limitation in people below the age of 45 years, the second most frequent reason for visiting physicians, the fifth most frequent cause of hospitalization, and the third most frequent ailment requiring surgical intervention.

Mechanisms of low back pain are not well understood. The term “Facet Syndrome” describes a mechanism of low back pain and instability originating from the peculiarities of the facet joints. In 1963, Hirsh demonstrated that injecting hypertonic saline in the region of the facet joints could simulate low back pain.

The superior and inferior processes of successive vertebral bodies form the facet joints. Also known as the apophyseal or zygapophyseal joints, the facet joints are innervated by the dorsal primary ramus of the nerve root. The medial branch of the dorsal ramus, also known as the facet nerve, is most closely associated with the facet joint, and each joint receives innervation from the facet nerves originating from the spinal levels above and below the facet joint. Facet Syndrome may occur at all levels of vertebrae, including cervical, thoracic and lumbar regions.

A typical case of Facet Syndrome displays localized back pain that is aggravated on hyperextension but not on flexion. Furthermore, there is local tenderness over the painful joints. Facet Syndrome may also entail referred pain into the groin, hip or thigh, and below the knee. Prolonged sitting and standing exasperate the pain. Range of motion may be decreased in all planes.

The main diagnostic test for determining whether facet joint pathology is the cause of low back pain has been by the injection of local anesthetic into the facet joint or onto the facet nerve. Significant relief of pain results in a positive Facet Syndrome diagnosis.

Interventional method of treatment to cure or relieve symptoms of Facet Syndrome have developed since the 1970's. Early treatments described inserting a long knife laterally to the location of the facet joint, cutting the facet nerve and thereby interrupting the sensory nerve supply to the joint. Further investigation revealed the facet nerve as the critical target of Facet Syndrome treatment.

Presently, the most common treatment for Facet Syndrome is radiofrequency facet nerve neurotomy. This treatment involves the insertion of a probe to a facet nerve and application of an electrical current through the probe. A generator that is connected to the probe produces the electrical current typically with a frequency between 460 kHz-500 kHz, which is in the radiofrequency range (3 kHz-300 GHz). The transmission of radiofrequency electrical current to the facet nerve causes heat to be produced, lesioning the facet nerve. Consequently, the sensory nerve supply is interrupted. U.S. Pat. No. 4,411,266 of Cosman, issued Oct. 25, 1983 teaches a probe that can be used for the treatment. A signal generator that can be used for this treatment is disclosed in U.S. patent application Ser. No. 10/122,413 of Shah and Baylis, filed on Apr. 16, 2002 incorporated herein by reference. At the time of the current disclosure, probes and radiofrequency generators manufactured by Baylis Medical Company, Inc., Montreal (Canada) are commercially available.

The facet nerve neurotomy procedure involves percutaneous insertion of a probe to a facet nerve, one or two levels caudal to the level being denervated. The probe is then advanced to the dorsal surface of the transverse process just caudal to the most medial end of the superior edge of the transverse process at the L1-L4 spinal levels. These anatomical landmarks can be identified with standard fluoroscopy. In order to denervate a single joint, both the facet nerve related to it laterally and the facet nerve from the next rostral segment are treated.

Once the position of the probe appears correct, two tests are typically conducted to confirm proximity to the targeted facet nerve and confirm that the probe is not in proximity to other nerves. To assess proximity to the facet nerve, an electrical stimulation is applied to the probe using a frequency that excites sensory nerves, typically 50 Hz with a current of up to 1 mA. A positive stimulation result reproduces the patient's pain, without producing other sensory responses in the lower extremity or buttocks. To confirm that the probe is not in proximity to an untargeted nerve, motor stimulation is achieved typically at a frequency of 2 Hz and a current of 3-5 mA. In this test, a lack of elicited muscle twitch in the lower limbs confirms that the probe is not at an undesired location near a spinal nerve. These tests indicate whether the probe is in proximity to the target nerve, and not in proximity to an undesired nerve. In the case of negative stimulation results, where there is a failure to produce the patient's pain or there is clear sensory or motor stimulation of the lower extremities, lesioning is not performed. Rather, the probe is repositioned and testing reperformed.

By passing continuous radiofrequency electrical current through the probe, a lesion is created as the temperature of the adjacent tissue rises to 60-90° C. and is held for 60 to 90 seconds.

Another treatment for Facet Syndrome includes the application of pulsed radiofrequency electrical current, which also passes radiofrequency electrical current to the facet nerve. This treatment differs from continuous radiofrequency in that it does not produce heat for electrocoagulation, but modifies the function of nervous tissue. Another treatment is the application of a pharmacological agent, such as nerve block. Upcoming techniques also include cryotherapy which delivers cryo- or cold energy to the facet nerve.

A challenging aspect for all Facet Syndrome treatments is the accurate localization of the facet nerve. The uncertainty in positioning of the probe within effective proximity of the facet nerve may be one reason for the variable success rates of these procedures. Prior art uses the patients' subjective feedback and practitioner's subjective observations to determine proximity to the targeted facet nerve. Furthermore, there is a need for confirmation of successful treatment of the facet nerve immediately following the procedure. It is therefore desirable to provide a patient-independent measurement of proximity and immediate confirmation of successful treatment of the facet nerve.

Prior art devices for facet nerve localization have not been used in conjunction with physiological sensors for accurate placement of the treatment probe or electrode. U.S. Pat. No. 6,325,764 of Griffith et al., issued Dec. 4, 2001 and U.S. Pat. No. 5,853,373 of Griffith et al., issued Dec. 29, 1998 describe a nerve locating apparatus and method for injecting anesthetic. In these inventions, the physician moves a stimulating electrode closer to a targeted motor nerve by attempting to maximize a visually observed muscle twitch. However, this apparatus and method measures the muscle twitch intensity based on subjective judgement of the physician. Furthermore, if the innervated muscle is subcutaneous or substantially small, the corresponding twitch or variation thereof may not be visually discernible. Confirmation of successful anesthesia administration of the targeted nerve is displayed by a lack of observed muscle twitch in response to electrical stimulation. However, the detection of a response is once again a subjective judgement of the physician.

Stimulation devices for depolarizing and hyperpolarizing the facet nerve have been described by U.S. Pat. No. 6,014,588 of Fitz, issued Jan. 11, 2000 and U.S. Pat. No. 6,314,325 of Fitz, issued Nov. 6, 2001. These devices use percutaneous electrodes to sense the electrical activity of muscles, or electromyography activity, surrounding a facet nerve for the purpose of minimizing the intensity of the stimulation. In these inventions, muscle activity is not used for placement of the electrode or for confirming treatment, but used instead to modulate stimulation generation.

In 2000, Dreyfus et al reported on the efficacy and validity of radiofrequency neurotomy for chronic lumbar facet joint pain. They suggest that a contributor to poor procedural efficacy is misdiagnosis. For example, it is possible that the facet joint is already denervated due to a concomitant or alternative pathology. Therefore, radiofrequency neurotomy would not relieve pain because the target nerve pathway does not exist and is not the cause of pain. To eliminate this error Dreyfus used electromyography of the L2-L5 bands of multifidus to assess the presence or absence of denervation before the neurotomy procedure. Six weeks after the neurotomy procedure the patients underwent another electromyogram to determine the presence or absence of denervation, and thus the success of the neurotomy procedure. This study improved procedural efficacy by reducing misdiagnosis. However, proximity of the electrode to the target nerve during the neurotomy procedure relied on subjective observation by the practitioner of muscle twitch elicited by nerve stimulation and subjective association of electrode position with radiological landmarks.

The clinical success rate of radiofrequency lumbar facet nerve neurotomy range from 9% (Lora & Long, 1976) to 83% (Ogsbury et al., 1977). According to Hall, the wide range of success rate is thought to be chiefly due to variability in positioning the electrode and the resulting lesion relative to the target nerve. This positioning can be especially challenging in the case of elderly patients who show osteoporosis leading to poor fluoroscopy visualization. An improvement in technique and apparatus for locating the facet nerve may increase the success rate of this procedure and eliminate improper probe positioning as a reason for poor success.

### SUMMARY OF THE INVENTION

A goal of the invention is to aid in the placement of a probe electrode to close proximity of a nerve requiring treatment, such as a facet nerve, by providing objective muscle activity measurements of a muscle innervated by the nerve.

A further goal is confirmation of successful nerve treatment, by objectively measuring the reduction in activity of the denervated muscle.

In accordance with an aspect of the invention, there is provided an apparatus that includes a generator to produce electrical current (e.g. radiofrequency) through a probe that is introduced into the body to near the facet nerve, accompanied by a physiological sensor and signal measuring device to measure and analyze multifidus muscle activity.

The invention provides an apparatus for localization of a target nerve. The apparatus includes a generator for the production of electrical current at physiological stimulation frequencies, one or more probe electrodes to transmit the electrical current to the target nerve, one or more physiological sensors for determining the proximity of the one or more probe electrodes to the target nerve, and a measuring device for reporting the physiological sensor signals to indicate the proximity.

The present invention provides a method for nerve proximity detection and optionally, treatment and confirmation of treatment of the nerve. The method comprises the steps of positioning at least one physiological sensor to detect muscle activity, positioning the probe, stimulating the nerve with an appropriate electrical current to elicit a response from the muscle, detecting the resulting muscle activity, and determining the proximity of the probe in response to the detected muscle activity. Optionally, the elicited activity may be maximized by repositioning the probe and re-testing. In a further option, nerve treatment may be performed and, as desired, a measure of the success of treatment by measuring the change in muscle activity elicited by a motor nerve stimulating current may be performed.

The present invention additionally provides a method of determining a measure of the success of a treatment of a nerve. By positioning at least one physiological sensor to detect an activity of a muscle innervated by the nerve, placing an electrically conductive probe proximal to the nerve, stimulating the nerve with an appropriate electrical frequency for eliciting a detectable response from the muscle, and detecting the muscle activity, the measure of success of the treatment in response to the detected muscle activity may be determined.

### BRIEF DESCRIPTION OF THE DRAWINGS

In order that the invention may be readily understood, embodiments of the invention are illustrated by way of examples in the accompanying drawings, in which:

FIG. 1 is an illustration of an apparatus setup in accordance with the invention;

FIG. 2 is a diagram of a probe of FIG. 1; and

FIG. 3 is a flow chart outlining a method of localization treatment and confirmation of treatment of the facet nerve in accordance with the invention.

### DETAILED DESCRIPTION OF THE INVENTION

In accordance with an aspect of the invention, a medical apparatus is provided for localizing a nerve in a patient particularly a facet nerve, though persons skilled in the art will appreciate that other nerves may be localized as desired. The medical apparatus comprises a generator for producing electrical current; one or more probes electrically connected to the generator for transmitting the electrical current to the nerve; one or more physiological sensors for detecting the activity of the muscle innervated by the nerve of the patient resulting from the transmitted electrical current; and a measuring device coupled to the one or more sensors for analyzing and preferably recording the muscle activity.

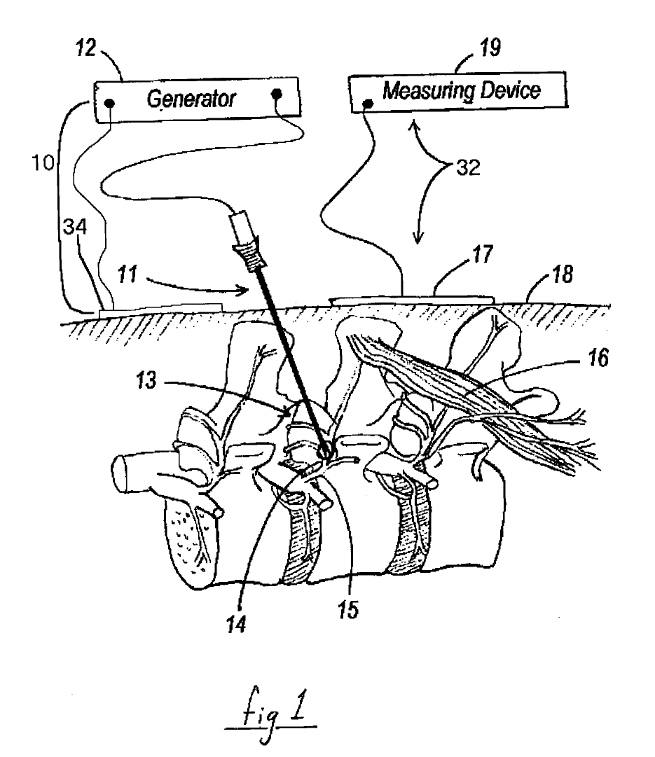

FIG. 1 depicts an exemplary medical apparatus, namely a radiofrequency neurotomy apparatus 10 in accordance with this aspect of the invention, using surface electromyography as the physiological sensor. Apparatus 10 comprises a probe 11 in electrical communication with an electrical current generator 12. Apparatus 10 further comprises a physiological sensor 32 comprising one or more surface electrodes 17 in electrical communication with a measuring device 19 for signal analysis and, preferably, recording. Probe 11 is shown percutaneously inserted into a patient's back and advanced to the dorsal surface of a transverse process in accordance with a method of treatment described further below. Insertion and advancement is typically aided by anatomical landmarks visualized by fluoroscopy as is well known to those skilled in the art. A facet nerve (or medial branch) 14 branches off the dorsal ramus nerve 15 which innervates the facet joint capsule 13 and the multifidus muscle 16. Surface electrodes 17 are shown positioned on skin 18 overlying a desired multifidus muscle 16, according to anatomical landmarks stated by DeFoa et al (1998).

The preferred generator 12 produces an electrical output in the physiological stimulation frequency range (0.5-200 Hz) for application to a patient by the probe 11. The preferred generator 12 also generates radiofrequency electrical current, for example, in the range of 3 kHz-300 GHz, preferably at least in the range between 460 kHz-500 kHz for delivery to the patient by the probe 11. An exemplary embodiment of a generator is the Pain Management Generator, model PMG-115, commercially available from Baylis Medical Company Inc., Montreal, PQ (Canada).

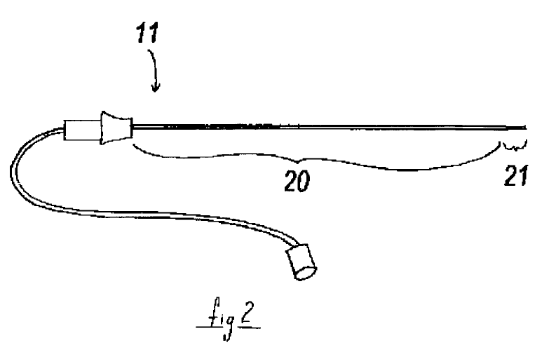

FIG. 2 shows one arrangement of the probe 11 in the current invention. Probe 11 comprises a shaft 20 insulated with one or more electrical insulators such as polytetrafluoroethylene (PTFE) and an active tip 21 at a distal end of the probe that is not insulated, and acts as an electrode. The electrical output delivered to the patient passes from the active tip 21 to a dispersive electrode 34 placed on the patent's skin.

In another embodiment (not shown), a cannula sheathing an uninsulated probe may be used to transmit electrical current. In this embodiment, the cannula includes an insulated shaft with an uninsulated tip that acts as the active tip.

In accordance with a preferred method aspect of the invention, the same probe is used to localize and treat the facet nerve. Suitable cannula and probes for this invention are commercially available from Baylis Medical Company, Inc., Montreal (Canada).

Physiological sensor 32 is used to measure the activity of multifidus muscle 16, in response to a stimulation current transmitted by probe 11 that excites facet nerve 14. A preferred arrangement of physiological sensor 32 is to place one or more surface electromyography electrodes 17 to target a specific multifidus muscle 16. The electrodes may be of conventional design, such as those commercially available from Delsys Inc., Boston, Mass. (USA).

A further arrangement of physiological sensor 32 includes one or more percutaneous electromyography needle electrode(s) (not shown) which may be placed to measure the activity of the multifidus muscle. Another embodiment of physiological sensor 32 comprises a motion sensor (not shown) to detect movement of multifidus muscle 16. Yet a further arrangement of physiological sensor 32 includes a percutaneous temperature sensor to measure the increase in temperature due to activity of the multifidus muscle 16.

Measuring device 19 is preferably used to amplify, analyze and record signals from physiological sensor 32. A preferred embodiment of measuring device 19 for use with electromyography electrode sensors is an electromyogram unit (not shown) comprising a multi-channel amplifier, data acquisition analog-to-digital card and digital signal analysis software, such as those commercially available from Deisys Inc., Boston, Mass. (USA). This unit amplifies electromyogram signals and converts the signal to digital format for storage and real-time analysis.

In a different embodiment, measuring device 19 is a waveform display device that displays the physiological sensor signal waveform. Commercially available devices such as an oscilloscope or bedside electrocardiogram unit may be used as is apparent to those skilled in the art.

A further embodiment of apparatus 10 comprises a communication link between the generator 12 and measuring device 19 to a correlating device such as a processor configured for receiving and analyzing signal information from generator 12 and measuring device 19 (not shown) in accordance with techniques well known to persons skilled in the art. Such an embodiment may by configured as an integrated system or single device (not shown). This embodiment may be configured to enable the time and amplitude correlation of the stimulation current output from the generator 12 and the activity of multifidus muscle determined by the physiological sensor 32. Detailed analysis of proximity to the nerve, association of signals and reduction of signal noise may be achieved by correlating time and amplitude of the signals.

Persons skilled in the art will further appreciate that the invention may be utilized in conjunction with other interventional pain management methods, including pharmacological nerve blockade, pulsed radiofrequency or cryotherapy application. The probe may be configured for selectively delivering to the nerve at least one of continuous radiofrequency electrical current, pulsed radiofrequency electrical current, pharmalogical nerve blockade and cryotherapeutic energy.

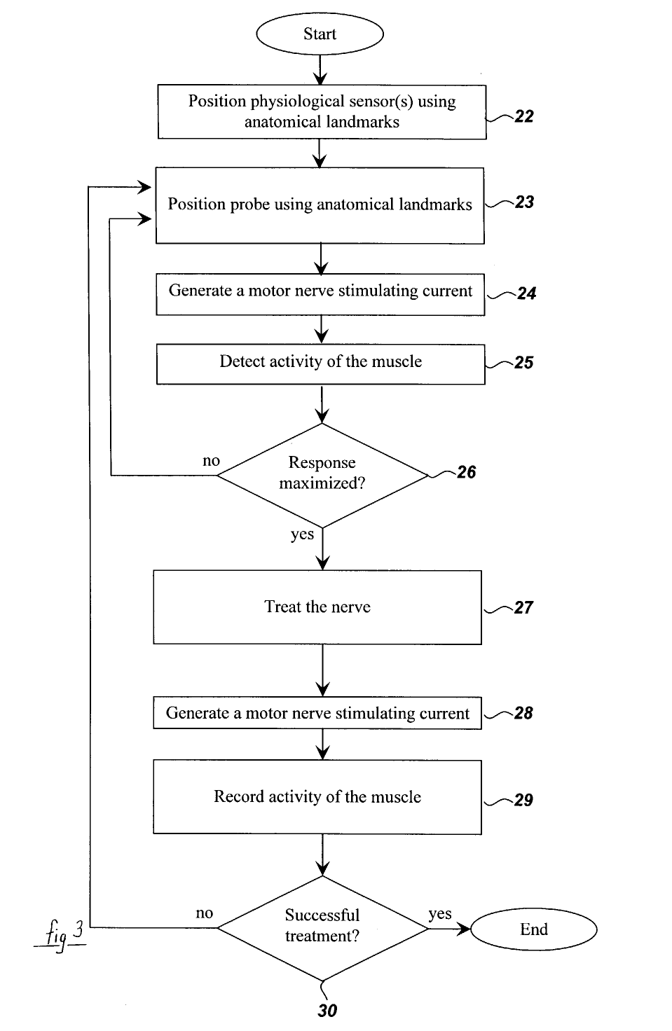

FIG. 3 shows a flow chart illustrating steps of a method of nerve proximity detection, treatment and confirmation of treatment in accordance with the invention for an exemplary facet nerve.

At step 22, physiological sensors 32 are placed on a patient to detect multifidus muscle activity in a desired area to be treated in accordance with anatomical landmarks.

Probe 11 is inserted and advanced according to established anatomical landmarks to place probe 11 near a facet nerve for treatment. Placement can be aided with the use of fluoroscopy as is well known in the art (step 23).

At step 24, a motor nerve stimulating current is generated by electric generator 12, typically with a frequency of 2 Hz and current 3-5 mA. Multifidus muscle activity is detected by physiological sensors 32 and communicated to measuring device 19 (step 25). The activity is preferably recorded to facilitate future analysis.

At step 26, a determination is made as to whether multifidus muscle activity response is maximized upon a review of the activity provided by measuring device 19 or a workstation coupled thereto (not shown). If insufficient response is detected or another reading desired, steps 23-25 may be selectively repeated though typically only steps 23-25 are likely to be repeated.

Optionally, though not shown, other tests may be performed as are well known to those skilled in the art. For example, visual monitoring of leg muscle twitch after motor stimulation may be performed to ensure the probe is not in proximity to an undesired motor nerve. As well, an optional test may include attempting to reproduce the patient's pain by sensory stimulation though this is not preferred.

Once the probe placement is determined, the facet nerve 14 is treated (step 27). Treatment may include, for example, applying a continuous radiofrequency electrical current to the active tip 21 to raise tissue temperature to 60-90° C. for 60 to 90 seconds to coagulate the facet nerve 14. Alternatively, and with a suitably constructed probe 11, other interventional pain management techniques may be used. Such techniques include the application of pharmacological nerve blockade, pulsed radiofrequency electrical current, or cryotherapy.

Steps 28 and 29 show similar monitoring steps illustrated at steps 24 and 25 to observe diminished multifidus muscle response as compared to the response recorded before the treatment in order to determine successful facet nerve treatment. If the treatment is not to the practitioner's satisfaction (step 30), prior steps may be selectively repeated, commencing at step 23, for example. It is apparent that repeated steps may start at step 22 or 27 as well.

It will be appreciated by persons skilled in the art that this apparatus and method may be used to treat Facet Syndrome at various vertebral levels. The invention could also be applied at other locations in the body. The method and apparatus of the current invention provides an objective aid in localization of the probe to the targeted nerve. Further, confirmation of neurotomy is assisted through immediate post-treatment success determination. Lastly, the method and apparatus of the current invention eliminates the necessity of reproducing the patient's pain to confirm successful positioning.

The embodiment(s) of the invention described above is (are) intended to be exemplary only. The scope of the invention is therefore intended to be limited solely by the scope of the appended claims.

We claim:

1. An apparatus for localization of a target nerve comprising:
i. a generator for the production of electrical current at physiological stimulation frequencies;
ii. one or more probe electrodes for coupling to the generator to transmit the electrical current to the target nerve;
iii. one or more physiological sensors for determining the proximity of the one or more probe electrodes to the target nerve, the sensors sensing and signaling multifidus muscle activity resulting from the transmission of electrical current to the target nerve by the probe electrodes; and
iv. a measuring device for reporting the physiological sensor signals to indicate the proximity.

2. The apparatus of claim 1, wherein the probe comprises an insulated shaft and an electrically conductive tip portion at a distal end of the shaft.

3. The apparatus of claim 1 wherein the probe is configured for treating the nerve.

4. The apparatus of claim 3, wherein the probe is configured for the selective delivery to the nerve of at least one of:
continuous radiofrequency electrical current for electrocoagulation;
pulsed radiofrequency electrical current for nerve function modification;
pharmacological nerve blockade; and
cryotherapeutic energy for nerve function modification.

5. The apparatus of claim 1, wherein at least one physiological sensor comprises an electromyography electrode to sense multifidus muscle electrical activity.

6. The apparatus of claim 1, wherein at least one physiological sensor comprises a motion sensor to sense muscle movement.

7. The apparatus of claim 1, wherein at least one physiological sensor comprises a percutaneous temperature sensor to sense changes in muscle temperature due to activity.

8. The apparatus of claim 1, wherein the measuring device comprises an electromyography unit capable of amplifying, analyzing and recording electromyogram signals.

9. The apparatus of claim 1, wherein the measuring device comprises a waveform display device to display the physiological sensor signal waveform.

10. The apparatus of claim 1, comprising a correlating device coupled to receive communications from the generator and measuring device whereby to correlate the generator stimulation and physiological sensor signals.

11. The apparatus of claim 1 wherein the apparatus is further for determination of a success of a treatment of the nerve and the measuring device is configured to indicate a measure of the success of the treatment in response to the said muscle activity.

12. A method of localization of a target nerve comprising the steps of:
i. positioning at least one physiological sensor to detect an activity of a muscle innervated by the target nerve;
ii. positioning an electrically conductive probe proximal to the target nerve;
iii. stimulating the target nerve with an appropriate electrical current to elicit a detectable response from the muscle innervated by the target nerve;
iv. detecting the muscle activity; and
v. determining the proximity of the probe to the target nerve in response to the detected muscle activity.

13. The method of claim 12 comprising the step of (vi) selectively repeating at least some of steps (ii) to (v) in order to maximize the muscle activity.

14. The method of claim 13 comprising the step of (vii) treating the nerve.

15. The method of claim 14, comprising the steps of:
(viii) stimulating the nerve with an appropriate electrical frequency to elicit a further detectable response;
(ix) detecting said further response; and
(x) determining a measure of a successful treatment in accordance with said further detected response.

16. The method of claim 15 comprising the step of (xi) selectively repeating at least one of steps (ii) to (x).

17. The method of claim 14, wherein step (vii) comprises the application to the nerve of at least one of:
continuous radiofrequency electrical current for electrocoagulation;
pulsed radiofrequency electrical current for nerve function modification;
pharmacological nerve blockade; and
cryotherapy for nerve function modification.

18. A method of determining a measure of the success of a treatment of a nerve comprising the steps of:
i. positioning at least one physiological sensor to detect an activity of a muscle innervated by the nerve;
ii. placing an electrically conductive probe proximal to the nerve;
iii. stimulating the nerve with an appropriate electrical frequency for eliciting a detectable response from the muscle;
iv. detecting the muscle activity; and
v. determining the measure of success of the treatment in response to the detected muscle activity.

19. The method of claim 18 comprising the step of (vi) selectively repeating steps (ii) to (v) in order to maximize the muscle activity.

20. The method of claim 20 comprising the step of (vii) treating the nerve.

21. The method of claim 21, comprising the step of:
(viii) selectively repeating steps (ii) to (v) in order to determine a further measure of a successful treatment.
